(19) United States
(12) Patent Application Publication
(10) Pub. No.: US 2017/0326186 A1
(43) Pub. Date: Nov. 16, 2017

(54) STRAINS OF LACTOBACILLUS AND BIFIDOBACTERIA FOR THE MAINTENANCE OF A LONG-LASTING HOMEOSTASIS CONDITION IN A HUMAN BODY

(71) Applicant: PROBIOTICAL S.p.A., Novara (NO) (IT)

(72) Inventor: Giovanni MOGNA, Novara (NO) (IT)

(21) Appl. No.: 15/528,044

(22) PCT Filed: Nov. 26, 2015
(86) PCT No.: PCT/IB2015/059146

§ 371 Date: May 18, 2017

(30) Foreign Application Priority Data
Nov. 26, 2014 (IT) MI2014A002035

(51) Int. Cl. A61K 35/747; A23L 33/135; A61K 35/745; C12N 1/20; C12R 1/225

(52) U.S. Cl. CPC A61K 35/747; C12N 1/20; C12R 1/225; A61K 35/745; A23L 33/135; A23V 2200/3202; A23V 2200/3204; A23V 2200/324

(57) ABSTRACT

Selected strains of bacteria belonging to the genus Lactobacillus and Bifidobacteria being able to maintain a long-lasting homeostasis condition in a human body are described. Furthermore, a composition is also described, including a food composition, a supplement product, a composition for a medical device or a pharmaceutical composition for use in the treatment of a long-lasting homeostasis condition in a human body; or for use in the treatment of renal failure, preferably acute or chronic; or for use in reducing uremic toxins, preferably of bacterial origin, such as indole and/or cresol.

The present invention relates to selected strains of bacteria belonging to the genus Lactobacillus and Bifidobacteria being able to maintaining a long-lasting homeostasis condition in a human body. Furthermore, the present invention relates to a composition, for example a food composition, a supplement product, a composition for a medical device or a pharmaceutical composition for use in the treatment of a long-lasting homeostasis condition of a human body; or for use in the treatment of renal failure, preferably acute or chronic; or for use in reducing uremic toxins, preferably of bacterial origin, such as indole and/or cresol.

Homeostasis is the attitude of living beings to maintaining, around a predetermined level, the value of some internal parameters, which are continuously disturbed by various external and internal factors. A network of control systems is designated for the ordered set of the sub-systems forming the human body, the simultaneous action of which regulates the flow of energy and metabolites, in order to keep unchanged or almost unchanged the Internal environment, regardless of the changes of the external one.

Self-regulation of living organisms is a major concept of modern biology. All the body's systems contribute for the maintenance of the homeostasis, but the main control center is the central nervous system, which establishes the most suitable kind of response (endocrine, immune etc.). Among the systems of the body, a very important system is represented by the immune system, which, along with the endocrine one, plays a crucial role in the body's defense against external stimuli represented by changes of the external environment.

Aging is known to be a process characterized by a remodeling of the immune system and a reduction of the functionality of the Immune response, and is related to an increased vulnerability to respiratory tract infections and a greater risk of death.

These age-related changes should to be attributed to the Immunosenescence phenomenon, an occurrence mainly caused by the continuous exposure, throughout the life span, to antigens and stressors such as, for example, the oxidative stress.

This results in an overall ‘deterioration’ of the Immune system, in which the increase of proinflammatory cytokines plays a crucial role: these, in fact, contribute to the Inflammaging process, which is meant as the phenomenon of chronic inflammation leading to the aging of the body.

Furthermore, the aging process is characterized by alterations in the redox homeostasis and progressive increase of the oxidative stress. This is involved in the transduction of the cell signaling, inflammatory response and tissue damage, causing metabolic and energetic changes, which modify some cell functions, such as the proliferation, the programmed cell death (apoptosis) and the homeostasis.

Reactive oxygen species (ROS) are also involved in the pathogenesis of several age-related diseases such as atherosclerosis, type II diabetes, neurodegeneration, osteoporosis, al of which share a strong inflammatory and immunological component. Furthermore, the oxidative stress and ROS are active inducers of apoptosis, the alterations of which, during aging, could account for some of the most crucial aspects of immunosenescence such as the build-up of expanded megaclones of memory cells, the shrinkage of T lymphocyte repertoire and the increase of autoimmune phenomena.

Cellular and molecular mechanisms related to the ability of the body to suitably react to the oxidative and inflammatory stress are thus likely to play a pivotal role in promoting the human longevity and avoiding/delaying the onset of the main age-related diseases. Furthermore, during aging a general reduction of plasma zinc levels is observed, leading to severe consequences for the immune system. Therefore, it thus remains the importance to avoid zinc deficits in advanced age and, thus, the need to intake it at proper doses and, mainly, to intake it in a form allowing the best bioavailability thereof, taking into consideration that the elderly population often suffers from intestinal malabsorption problems. Therefore, there is still a need for having a solution to the above problems which would allow to counteracting the deterioration of the immune system, delaying the aging process and maintaining a long-lasting homeostasis condition.

A good renal functionality also contributes to the maintenance of a long-lasting homeostasis condition. Many factors concur to compromise the renal functionality, among which a blood build-up of nitrogenous substances, in particular urea, due to the kidney inability to excrete them. This build-up results in uremia, which represents the final step of renal failure (acute or chronic).

When kidneys are no longer able to exert their functions, the renal failure step occurs, which, depending on the deterioration extent, can be acute or chronic. In most of renal diseases, the kidney function progressively deteriorates. The unsuccessful elimination of waste products (uremic toxins, such as indole and cresol which are bacterial uremic toxins from microbiota, for example from bacteria E. coli) held by the body, exerts toxic effects in almost all the body's organs. The main subjective manifestations of the uremic condition are: nausea, weakness, sleepiness, difficulties in feeding and performing the normal daily activities.

Therefore, it is fundamental to being able to intervene and reduce uremic toxins for maintaining a good renal functionality as well as a good homeostasis condition.

The Applicant found that in order to maintaining a long-lasting homeostasis condition it is important to stimulate the immune system so that a controlled and moderate endogenous production or secretion, which contributes to the maintenance of specific and well-established cytokines within predetermined ranges of values, takes place.

Following to an intense and extended research and development activity, the Applicant fulfilled the above-cited need by selecting specific strains of bacteria belonging to the genus Lactobacillus and Bifidobacteria being able to act on two fronts: against immunosenescence and against the Inflammaging phenomenon.

The Applicant, for the first time, found that specific strains of bacteria selected from those belonging to the genus Lactobacillus and Bifidobacteria which, for a given set of cytokines measurable in specific human cell lines, have values within specific ranges, are able to counteracting the deterioration of the immune system, delaying the aging process and maintaining a long-lasting homeostasis condition by modulating the production of cytokines having both anti-inflammatory activity and proinflammatory activity (IFN-gamma).

It is an object of the present invention a strain of bacteria belonging to the genus Lactobacillus or Bifidobacteria, said strain being characterized by having the following features:

(i) a capability to modulate the immune system by modulating the production of the anti-inflammatory cytokine, such as IL-4, to a value comprised from 2.5 to 4.5 folds, relative to the baseline value set equal to 1; preferably from 3 to 4 folds, relative to the baseline value set equal to 1; and
(ii) a capability to modulate the immune system by modulating the production of the proinflammatory cytokine, such as IL-12p70, to a value comprised from 0.85 to 1.05 folds, relative to the baseline value set equal to 1; preferably from 0.90 to 1 folds, relative to the baseline value set equal to 1; and
(iii) a capability to modulate the immune system by modulating the production of the proinflammatory cytokine, such as IFN-gamma, to a value comprised from 7 to 19.5 folds, relative to the baseline value set equal to 1; preferably from 8 to 18 folds, relative to the baseline value set equal to 1; and
(iv) a capability to modulate the immune system by modulating the production of proinflammatory cytokines, such as IL-17, to a value comprised from 0.90 to 1.40 folds, relative to the baseline value set equal to 1; preferably from 0.95 to 1.30 folds, relative to the baseline value set equal to 1; and
(v) an overall capability to modulate the ratio of proinflammatory/anti-inflammatory cytokines to give a value of the Th1/Th2 ratio comprised from 2.90 to 4.50; preferably to a value comprised from 3 to 4.

Further preferred embodiments of the present invention will be set forth and illustrated hereinafter in the following detailed description without wishing to limit in any way the scope of the present invention.

The Applicant conducted an intense research activity, during which detected, selected, isolated and characterized the following bacterial strains which are the object of the present invention.

The Applicant found that the bacterial strains belonging to the genus Lactobacillus or Bifidobacteria which fulfill al the above-cited conditions (i)-(v), upon administration to an organism for a period of time of at least 2 weeks, preferably from 4 to 8 weeks, are able to stimulate the immune system with a controlled and moderate endogenous production or secretion, which contributes to the maintenance of specific and well-established cytokines within predetermined ranges of values.

Advantageously, the compositions of the present invention containing at least a strain of bacteria belonging to the genus Lactobacillus or Bifidobacteria which fulfill all the above-cited conditions (i)-(v), are effectively applied for modulating the immune system, delaying the aging process and maintaining a long-lasting homeostasis condition, thus providing health benefits to the body.

In an embodiment the strains of bacteria belonging to the genus Bifidobacteria, which fulfil al the above-cited conditions (i)-(v), were isolated and selected from human fecal matter from ultra-centenarian subjects. These bacterial strains belong to the species Bifidobacterium longum.

Advantageously, the strains of bacteria belonging to the species Bifidobacterium longum are selected from the group comprising or, alternatively, consisting of: Bifidobacterium longum DLBL 07 DSM 25669, Bifidobacterium longum DLBL 08 DSM 25670, Bifidobacterium longum DLBL 09 DSM 25671, Bifidobacterium longum DLBL 10 DSM 25672, Bifidobacterium longum DLBL 11 DSM 25673, or mixtures thereof.

Said strains of bacteria were deposited on 16.02.2012 by the Company Probiotical SpA Via Mattei, 3-28100 Novara (NO) Italy, according to the Budapest Treaty at DSMZ-Deutsche Sammlung von Mikro-organismen und Zellkulturen GmbH, Germany.

The above-cited strains of bacteria are effectively used for preparing a pharmaceutical composition or a medical device or a supplement product or a food composition (briefly, hereinafter “the compositions of the present invention”), as described and claimed below.

A first embodiment is represented by a composition for an oral medical device, said composition comprises or, alternatively, consists of:

(a) at least a strain of bacteria belonging to the genus Lactobacillus or Bifidobacteria which fulfills all the above-cited conditions (i)-(v); for use in modulating the immune system, delaying the aging process, maintaining a long-lasting homeostasis condition, thus providing health benefits to the body, and promoting an enhanced intestinal functionality. Advantageously the component (a) of the composition comprises or, alternatively, consists of: Bifidobacterium longum DLBL 07 DSM 25669, Bifidobacterium longum DLBL 08 DSM 25670, Bifidobacterium longum DLBL 09 DSM 25671, Bifidobacterium longum DLBL 10 DSM 25672 and Bifidobacterium longum DLBL 11 DSM 25673.

Another embodiment is represented by a composition for an oral medical device, said composition comprises or, alternatively, consists of:

(a) at least a strain of bacteria belonging to the genus Lactobacillus or Bifidobacteria which fulfills all the above-cited conditions (i)-(v); and
(b) a specific mucoadherent gelling complex, consisting of exopolysaccharides (EPS) of bacterial origin produced in situ by the strain of bacterium Streptococcus thermophilus ST10 DSM25246 and a polysaccharide of plant origin; preferably tara gum; for use in modulating the immune system, delaying the aging process, maintaining a long-lasting homeostasis condition, thus providing health benefits to the body, and promoting an enhanced intestinal functionality. Advantageously, the component (a) of the composition comprises or, alternatively, consists of: Bifidobacterium longum DLBL 07 DSM 25669, Bifidobacterium longum DLBL 08 DSM 25670, Bifidobacterium longum DLBL 09 DSM 25671, Bifidobacterium longum DLBL 10 DSM 25672 and Bifidobacterium longum DLBL 11 DSM 25673.

Said composition for oral medical device is able to modulating the immune system, delaying the aging process, maintaining a long-lasting homeostasis condition, thus providing health benefits to the body, and promoting a good intestinal functionality.

Said component (a) can be one or more of the strains of bacteria belonging to the species Bifidobacterium longum selected from the group comprising or, alternatively, consisting of: Bifidobacterium longum DLBL 07 DSM 25669, Bifidobacterium longum DLBL 08 DSM 25670, Bifidobacterium longum DLBL 09 DSM 25671, Bifidobacterium longum DLBL 10 DSM 25672, Bifidobacterium longum DLBL 11 DSM 25673, or mixtures thereof (component (a)).

The composition contains a specific mucoadherent gelling complex (component (b)), consisting of EPS, exopolysaccharides and tara gum, being able to form a hydrogel within few minutes after ingestion due to its thixotropic features and thereby create a mechanical barrier effect against metabolites with proinflammatory activity and thus able to enhance the oxidative stress of the body, promoting the aging processes both at a macromolecular and cellular level.

In a preferred embodiment, the composition contains a mixture comprising or, alternatively, consisting of the five microorganisms (component (a)) which fulfil the conditions (i)-(v) Bifidobacterium longum DLBL 07 (DSM 25669), Bifidobacterium longum DLBL 08 (DSM 25670), Bifidobacterium longum DLBL 09 (DSM 25671), Bifidobacterium longum DLBL 10 (DSM 25672) and Bifidobacterium longum DLBL 11 (DSM 25673), isolated from centenarian subjects and being able to strengthen the barrier effect against gut microbes and metabolites related to aging, directly acting on the qualitative and quantitative composition of the intestinal microbiota.

Furthermore, there is also the microorganism Streptococcus thermophilus ST10 (in association with tara gum), which conversely is able to synthesizing in situ exopolysaccharides (EPS) and thereby increasing the viscosity of the surrounding environment. The intake of the above-mentioned bacterium ST10 provides the human gut of a source of molecules with gelling activity, thus exerting a synergistic action with tara gum and, thereby strengthening the mechanical barrier effect against metabolites with proinflammatory activity and thus able to increase the oxidative stress of the body, promoting the aging processes both at a macromolecular and cellular level.

In another embodiment the composition can further contain, in addition to the strains of bacterium (a) and the mucoadherent gelling complex (b), the strains Lactobacillus buchneri Lb 26 (DSM 16341) and/or Bifidobacterium lactis Bb1 (DSM 17850) (ProbioSel® and ProbioZinc®, respectively) which provide selenium and zinc in a form highly assimilable by the body, thus strengthening the defense mechanisms against the oxidative stress. Said bacterial strains were deposited at DSMZ Institute in Germany, by the Company Bioman S.r.l., Via Alfieri 18, 10100 Torino (Italy). Advantageously, the component (a) of the composition comprises or, alternatively, consists of Bifidobacterium longum DLBL 07 DSM 25669, Bifidobacterium longum DLBL 08 DSM 25670, Bifidobacterium longum DLBL 09 DSM 25671, Bifidobacterium longum DLBL 10 DSM 25672 and Bifidobacterium longum DBL 11 DSM 25673.

In another embodiment the composition can further contain, in addition to the strains of bacterium (a), the mucoadherent gelling complex (b) and the strains Lactobacillus buchneri Lb 26 (DSM 16341) and/or Bifidobacterium lactis Bb1 (DSM 17850), the strain Bifidobacterium lactis BA05 (DSM 18352), being able to synthesize folates and counterbalance, in this way, the progressive deficit of this metabolite during aging. Advantageously the component (a) of the composition comprises or, alternatively, consists of Bifidobacterium longum DLBL 07 DSM 25669, Bifidobacterium longum DLBL 08 DSM 25670, Bifidobacterium longum DLBL 09 DSM 25671, Bifidobacterium longum DLBL 10 DSM 25672 and Bifidobacterium longum DLBL 11 DSM 25673.

In the light of their overall mechanism of action, the compositions being object of the present invention synergistically combine a first effect deriving from the maintenance of specific and well-established cytokines, and subpopulations thereof, such as IL-4, IL-12, IFN-gamma, IL-17 and the Th1/Th2 ratio of proinflammatory/anti-inflammatory cytokines, within predetermined ranges of values as specified above in items (i)-(v), along with a second effect deriving from the establishment and maintenance of an effective mechanical barrier effect against gut microbes and metabolites more or less strongly related to a high oxidative stress and aging.

The tara gum present in the mucoadherent gelling complex is progressively degraded during its intestinal transit by the resident microbiota, thus progressively reducing its gelling ability of mechanical hindrance. The gradual reduction of the plant gum action is effectively counterbalanced by the gradual increase of exopolysaccharide (EPS) release in the intestinal lumen by the bacterial strain ST10, which exerts its effect mainly in the ileum and colon.

The synergistic combination of tara gum and exopolysaccharides (EPS) produced in situ ensures, in this way, the presence of gelling molecules throughout the gastrointestinal tract, maximizing and optimizing the mechanical barrier action specific of the product. The presence, production and maintenance of the hydrophilic gel in the lumen of the organ can thus be considered, for the first time, really complete, with a first area where the action of the plant gum is maximum and a second area where the action of exopolysaccharides (EPS) is maximum.

With regard to the above, aging is known to be a process characterized by a remodeling of the immune system and a reduction of the functionality of the immune response. These age-related changes should be attributed to the immunosenescence phenomenon, an occurrence mainly caused by the continuous exposure, throughout the life span, to antigens and stressors such as, for example, oxidative stress.

This results in a general “deterioration” of the immune system, in which the increase of proinflammatory cytokines plays a crucial role: in fact they contribute to the Inflammaging process, which is meant as a chronic inflammation phenomenon leading to the aging of the body.

Furthermore, the aging process is characterized by changes in the redox homeostasis and progressive increase of oxidative stress. The cellular and molecular mechanisms related to the ability of the body to suitably react to both oxidative and inflammatory stresses seem thus to play a crucial role in promoting the human longevity and avoiding or delaying the onset of the major age-related dysfunctions.

Another important aspect is the characteristic of the intestinal microbiota to qualitatively and quantitatively vary during aging. The gut bacterial population, in fact, can undergo changes in its composition due to immunological and mucosal barrier modifications. Such a microbiota variation in the elderly subject is mainly valuable when considering that the overgrowth of some bacterial species can result in deficit of calcium, iron and folates, which are elements required by the bacterial species for their growth.

From the above, it can be inferred the importance of taking into account in the aging a proper and suitable equilibrium of the intestinal microbiota, mainly relative to the progressive and, in most of the cases, irreversible loss of function of the normal barrier effect of the mucosa against metabolites with proinflammatory and pro-oxidant activities.

The mixture of the five microorganisms Bifidobacterium longum DLBL 07 (DSM 25669), Bifidobacterium longum DLBL 08 (DSM 25670), Bifidobacterium longum DLBL 09 (DSM 25671), Bifidobacterium longum DLBL 10 (DSM 25672) and Bifidobacterium longum DLBL 11 (DSM 25673)-component (a), isolated from centenarian subjects and able to strengthen the barrier effect against the gut microbes and metabolites related to aging, mediated by the mucoadherent gelling complex consisting of tara gum and exopolysaccharides (EPS), by directly acting on the qualitative and quantitative compositions of the intestinal microbiota. During aging, a general reduction of the ability to absorbing zinc at the intestinal level is observed, thus leading to a deficit condition with crucial consequences for thymus and immune system.

Zinc is, in fact, an element indispensable for the normal functioning of the immune system, as it exerts a polyvalent action influencing any aspect of the immune response. The zinc in human body, of about 2 grams, is distributed throughout the tissues, but mainly concentrates in the striated musculature (60%), bones (30%) and skin (4-6%). Only the hepatic zinc can be partially mobilized in the case of a time-limited deficit, but no specific reservoir of zinc exists, thereby a regular dietary intake is required. Approximately 10-40% of the zinc introduced with food is absorbed at the proximal intestine level. The absorbed amount varies depending on its chemical form, its blood concentration, the simultaneous presence in the intestinal lumen of microelements competing for the transport, chelating agents and the concentration of metallothionein synthetized by mucosal cells.

However, on the basis of the above, it remains the importance for avoiding, in the advanced age, zinc deficits and, thus, the need to intake it at proper doses and, mainly, to intake it in a form allowing the best bioavailability thereof, considering that the elderly population often suffers from intestinal malabsorption problems.

The selenium dietary supplementation is also fundamental for allowing the release of zinc from the intracellular compartments, wherein is sequestered, a frequent occurrence in the elderly population, and synergistically acting with Zinc itself for the antioxidant activity. Selenium is, indeed, a constituent of glutathione peroxidase, an enzyme with antioxidant activity, crucial for counteracting the oxidative stress.

In an embodiment, the strains of Lactobacillus buchneri Lb26 (DSM 16341) and Bifidobacterium lactis Bb1 (DSM 17850) (ProbioSel® and ProbioZinc®, respectively) are in the composition being object of the present invention in tyndallized form; in this form said strains are able to provide Selenium and Zinc in a form highly assimilable by the body useful to compensate for the deficit derived by the aging process, strengthening, in an ancillary manner, the defense mechanisms against oxidative stress, particularly important for people over 40-50 years. Such tyndallized bacteria, upon reaching the intestinal mucosa, release, close to enterocytes, zinc and selenium in organic form, which are thus directly absorbed through the intestinal mucosa and ready for entering the systemic circulation and exerting their effect on the organism.

The strain of bacterium Bifidobacterium lactis Bb1 (DSM 17850) is able to accumulate zinc inside the cell during its growth in a liquid medium. The dietary zinc accumulated inside the microorganism cell has an assimilability of more than 16-fold greater than zinc gluconate and 31.5-fold greater than zinc sulphate, as shown by a study in vitro conducted on Caco-2 cells, which mimic the Intestinal epithelium, in a Transwell system.

The strain of bacterium Lactobacillus buchneri Lb26 (DSM 16341) is able to accumulate selenium inside the cell. Said Selenium has an assimilability 5.9-fold greater than sodium selenite, 9.4-fold greater than selenium methionine and even 65-fold greater than selenium cysteine.

The high assimilability of the two trace elements zinc and selenium allows to counteracting in a very effective manner the deficits even at very low dosages.

The possibility for having zinc directly available at systemic level avoids all those problems related to the intake thereof in an elderly subject with difficulties of intestinal absorption, counterbalancing the deficit of this trace element.

Furthermore, the combination also with Selenium overcomes that above-cited problem concerning the typical sequestering by metallothioneins, in the advanced age, of zinc at intracellular level; selenium, in fact, promotes the release thereof from cells.

The further presence of the microorganism Bifidobacterium lactis BA05 (DSM 18352) in the composition of the present invention, strengthens the mechanical barrier effect described above and, additionally, is able to naturally synthesizing in situ folates (vitamin 89) and counterbalancing, in this way, the progressive deficit of this antioxidant metabolite during aging. Particularly, folates are known to exert a barrier effect against homocysteine, a molecule derived from a sulfur amino acid being able to inducing a strong production of free radicals and a consequent oxidative stress.

In the light of Its overall mechanism of action, the composition of the present invention is active against Inflammaging, primarily acting through the establishment and maintenance of an effective mechanical barrier effect against metabolites, which are more or less strongly related to a high oxidative stress and aging, and counterbalancing the deficit of specific micronutrients and folates having a crucial activity in adults over 50 for ensuring a healthy advance towards old age.

It can be thereby stated that the composition of the present invention establishes and maintains a barrier effect, mainly of mechanical type, against Inflammaging, resulting thus able to assist in facing the aging in a better health condition.

In the context of the present invention, the strains of bacteria belonging to the species Lactobacillus or Bifidobacteria which fulfill the conditions (i)-(v), such as the strains of bacteria belonging to the species Bifidobacterium longum, can be in said compositions of the present invention in the form of live cells and/or dead cells and/or as a metabolite thereof and/or as a cell derivative thereof and/or as a cellular or enzymatic component thereof.

The compositions of the present invention can be administered to al the categories of people without restrictions for maintaining a long-lasting homeostasis condition, assisting the extension of the life span of a subject; delaying and/or counteracting and/or reducing the biological processes of aging, for example the aging of the body and/or skin; reducing the aging processes leading to a loss of memory or visual memory and/or capacity to concentrate; inhibiting the production of Bacteroides through a non-specific (production of metabolites) and/or specific (production of bacteriocins) inhibition mechanism; stimulating the production of butyric Clostridia being able to produce butyrate which is capable to inhibit the phenomena leading to the onset of colitis, ulcerative colic, IBD (Inflammatory Bowel Disease) and Crohn's disease; inhibiting and/or reducing the production of Enterobacteria belonging to the Enterobacteriaceae family, in particular reducing the load of enterobacteria usually existing in a microbiota; modifying the intestinal microflora equilibrium in order to allowing the species Bifidobacterium longum to prevail; positively influencing the antioxidant activity, the immunomodulatory activity with the production of cytokines.

The strains of bacteria belonging to the species Bifidobacterium longum which fulfill all the above-cited conditions (i)-(v), being object of the present invention, can assist to contribute in the extension of the life span of a human being since said strains can significantly intervene due to their proteasome (UPS=ubiquitin-proteasome-system). The action of proteasome allows to counteracting the biological phenomena leading to the aging thus preserving the physical and/or mental condition of a human being.

Advantageously, the compositions of the present invention can comprise N-acetylcysteine (NAC) as such or a substance based on N-acetylcysteine (NAC) or a derivative thereof combined with at least a strain of bacteria which fulfill the conditions (i)-(v).

Therefore, it is contemplated in the present invention the use of N-acetylcysteine both as free and microencapsulated gasto-protected form (from 10 to 1000 mg/die) having a mechanical barrier effect for counteracting the adhesive abilities of E. coli to the intestinal wall. Furthermore, N-acetylcysteine stimulates the glutathione production and, thus, it has an antioxidant activity. The compositions of the present invention are able to preserve the activity of the proteasome. For this reason, they can be effectively administered to people for helping them in extending the life span.

The strains of bacteria belonging to the genus Lactobacillus or Bifidobacteria which fulfill al the conditions (i)-(v) are in an amount comprised from 0.1 to 65% by weight, preferably from 0.5 to 15% by weight even more preferably from 1 to 10% by weight, relative to the total weight of the composition. However, said percentage relative to the total weight of the composition, depends on the product category of the composition to be prepared. For example, the amount of said bacteria in a capsule is preferably greater than 40%.

The compositions of the present invention contain a bacterial load having a concentration comprised from 1×10⁶ to 1×10¹¹ UFC/g, preferably from 1×10⁸ to 1×10¹⁰ UFC/g.

The compositions can contain bacteria in a concentration comprised from 1×10⁶ to 1×10¹¹ UFC/dose, preferably from 1×10⁸ to 1×10¹⁰ UFC/dose. The dose can be comprised from 0.2 to 10 g, for example 0.25 g, 1 g, 3 g, 5 g, or 7 g.

The bacteria used in the present invention can be in solid form, particularly as powder, dehydrated, spray or freeze-dried powder.

The food composition or supplement product or medical device or pharmaceutical composition can further comprise also some prebiotic fibers and carbohydrates with bifidogenic activity such as for example inulin, fructo-oligosaccharides (FOS), galacto- and trans-galacto-oligosaccharides (GOS and TOS), gluco-oligosaccharides (GOSα), xylo-oligosaccharides, (XOS), chitosan-oligosaccharides (COS), soya-oligosaccharides (SOS), isomalto-oligosaccharides (IMOS), resistant starch, pectins, psyllium, arabinogalactans, glucomannans, galactomannans, xylans, lactosucrose, lactulose, lactitol and many other types of gums, preferably tare gum, acacia, locust, oat, bamboo fiber, citrus fruit fibers and, in general, fibers containing a soluble and an insoluble portion, in a variable ratio from each other.

Advantageously, said fiber is selected from the group comprising FOS, inulin and citrus fruit fibers, preferably in a weight ratio from 1:3 to 3:1.

The amount of prebiotic fibers and/or carbohydrates with bifidogenic activity, if any, is comprised from 0.5 to 75% by weight, preferably from 1% to 40% and even more preferably from 2 to 20% relative to the total weight of the composition. In this case the composition or supplement product has a symbiotic activity.

The compositions of the present invention can further comprise one or more physiologically acceptable additives or excipients as well as further comprise also other ingredients and/or active components such as vitamins, minerals, bioactive peptides, substances with antioxidant, hypocholesterolemic, hypoglycemic, anti-inflammatory activity, sweeteners in an amount by weight usually comprised from 0.001% to 10% by weight, preferably from 0.5 to 5% by weight, in any case depending on the kind of active component and any recommended daily dose thereof, relative to the total weight of the composition. The compositions of the present invention are prepared by techniques known and accessible to the skilled in the fled, which is able to use the known equipment and devices and the suitable production methods.

Table A relates to the strains tested by the Applicant in the context of the present invention.

TABLE A

| | | Comm. | Deposit | Deposit | Deposit | |
|---|---|---|---|---|---|---|
| No. | Name | abbreviation | institute | number | date | Owner |
| 1 | Lactobacillus casei | LF1i | CNCM I.P. | I-785 | 21 Jul. 1988 | Anidral Srl |
| 2 | Lactobacillus gasseri | LF2i | CNCM I.P. | I-786 | 21 Jul. 1988 | Anidral Srl |
| 3 | Lactobacillus crispatus | LF3i | CNCM I.P. | I-787 | 21 Jul. 1988 | Anidral Srl |
| 4 | Lactobacillus fermentum | LF4i | CNCM I.P. | I-788 | 21 Jul. 1988 | Anidral Srl |
| 5 | Lactobacillus fermentum | LF5 | CNCM I.P. | I-789 | 21 Jul. 1988 | Anidral Srl |
| 6 | Lactobacillus casei ssp. | LFH i | CNCM I.P. | I-790 | 21 Jul. 1988 | Anidral Srl |
| | pseudoplantarum | | | | | |
| 7 | Streptococcus thermophilus B39 | | BCCM LMG | LMG P-18383 | 5 May 1998 | Anidral Srl |
| 8 | Streptococcus thermophilus T003 | | BCCM LMG | LMG P-18384 | 5 May 1998 | Anidral Srl |
| 9 | Lactobacillus pentosus 9/1 ei | | BCCM LMG | LMG P-21019 | 16 Oct. 2001 | Mofin Srl |
| 10 | Lactobacillus plantarum 776/1 bi | LP 02 | BCCM LMG | LMG P-21020 | 16 Oct. 2001 | Mofin Srl |
| 11 | Lactobacillus plantarum 476LL 20 bi | LP 01 | BCCM LMG | LMG P-21021 | 16 Oct. 2001 | Mofin Srl |
| 12 | Lactobacillus plantarum PR ci | | BCCM LMG | LMG P-21022 | 16 Oct. 2001 | Mofin Srl |
| 13 | Lactobacillus plantarum 776/2 hi | | BCCM LMG | LMG P-21023 | 16 Oct. 2001 | Mofin Srl |
| 14 | Lactobacillus casei ssp. paracasei | LPC00 | BCCM LMG | LMG P-21380 | 31 Jan. 2002 | Anidral Srl |
| | 181A/3 aiai | | | | | |
| 15 | Lactobacillus belonging to the | LA 02 | BCCM LMG | LMG P-21381 | 31 Jan. 2002 | Anidral Srl |
| | acidophilus group 192A/1 aiai | | | | | |
| 16 | Bifidobacterium longum 175A/1 aiai | | BCCM LMG | LMG P-21382 | 31 Jan. 2002 | Anidral Srl |
| 17 | Bifidobacterium breve 195A/1 aici | | BCCM LMG | LMG P-21383 | 31 Jan. 2002 | Anidral Srl |
| 18 | Bifidobacterium lactis 32A/3 aiai | BS 01 | BCCM LMG | LMG P-21384 | 31 Jan. 2002 | Anidral Srl |
| 19 | Lactobacillus plantarum 501/2 gi | COAKTIV | BCCM LMG | LMG P-21385 | 31 Jan. 2002 | Mofin Srl |
| 20 | Lactococcus lactis ssp. lactis 501/4 ci | | BCCM LMG | LMG P-21388 | 31 Jan. 2002 | Mofin Srl |
| 21 | Lactococcus lactis ssp. lactis 501/4 hi | | BCCM LMG | LMG P-21387 | 15 Mar. 2002 | Mofin Srl |
| 22 | Lactococcus lactis ssp. lactis 501/4 ci | | BCCM LMG | LMG P-21388 | 31 Jan. 2002 | Mofin Srl |
| 23 | Lactobacillus plantarum 501/4 li | | BCCM LMG | LMG P-21389 | 15 Mar. 2002 | Mofin Srl |
| 24 | Lactobacillus acidophilus | LA08 | BCCM LMG | LMG P-26144 | 3 Nov. 2010 | Probiotical SpA |
| 25 | Lactobacillus paracasei ssp. | LPC10 | BCCM LMG | LMG P-26143 | 3 Nov. 2010 | Probiotical SpA |
| | paracasei | | | | | |
| 26 | Streptococcus thermophilus | GB1 | DSMZ | DSM 16506 | 18 Jun. 2004 | Anidral Srl |
| 27 | Streptococcus thermophilus | GB5 | DSMZ | DSM 16507 | 18 Jun. 2004 | Anidral Srl |
| 28 | Streptococcus thermophilus | Y02 | DSMZ | DSM 16590 | 20 Jul. 2004 | Anidral Srl |
| 29 | Streptococcus thermophilus | Y03 | DSMZ | DSM 16591 | 20 Jul. 2004 | Anidral Srl |
| 30 | Streptococcus thermophilus | Y04 | DSMZ | DSM 16592 | 20 Jul. 2004 | Anidral Srl |
| 31 | Streptococcus thermophilus | YO5 | DSMZ | DSM 16593 | 20 Jul. 2004 | Anidral Srl |
| 32 = | Bifidobacterium adolescentis | BA 03 | DSMZ | DSM 16594 | 21 Jul. 2004 | Anidral Srl |
| 56 | | | | | | |
| 33 | Bifidobacterium adolescentis | BA 04 | DSMZ | DSM 16595 | 21 Jul. 2004 | Anidral Srl |
| 34 | Bifidobacterium breve | BR 04 | DSMZ | DSM 16596 | 21 Jul. 2004 | Anidral Srl |
| 35 | Bifidobacterium pseudocatenulatum | BP 01 | DSMZ | DSM 16597 | 21 Jul. 2004 | Anidral Srl |
| 36 | Bifidobacterium pseudocatenulatum | BP 02 | DSMZ | DSM 16598 | 21 Jul. 2004 | Anidral Srl |
| 37 | Bifidobacterium longum | BL 03 | DSMZ | DSM 16603 | 20 Jul. 2004 | Anidral Srl |
| 38 | Bifidobacterium breve | BR 03 | DSMZ | DSM 16604 | 20 Jul. 2004 | Anidral Srl |
| 39 | Lactobacillus casei ssp. rhamnosus | LR 04 | DSMZ | DSM 16605 | 20 Jul. 2004 | Anidral Srl |
| 40 | Lactobacillus delbrueckii ssp. | LDB 01 | DSMZ | DSM 16606 | 20 Jul. 2004 | Anidral Srl |
| | bulgaricus | | | | | |
| 41 | Lactobacillus delbrueckii ssp. | LDB 02 | DSMZ | DSM 16607 | 20 Jul. 2004 | Anidral Srl |
| | bulgaricus | | | | | |
| 42 | Staphylococcus xylosus | SX 01 | DSMZ | DSM 17102 | 1 Feb. 2005 | Anidral Srl |
| 43 = | Bifidobacterium adolescentis | BA 02 | DSMZ | DSM 17103 | 1 Feb. 2005 | Anidral Srl |
| 57 | | | | | | |
| 44 | Lactobacillus plantarum | LP 07 | DSMZ | DSM 17104 | 1 Feb. 2005 | Anidral Srl |
| 45 | Streptococcus thermophilus | YO8 | DSMZ | DSM 17843 | 21 Dec. 2005 | Anidral Srl |
| 46 | Streptococcus thermophilus | YO9 | DSMZ | DSM 17844 | 21 Dec. 2005 | Anidral Srl |
| 47 | Streptococcus thermophilus | YO100 | DSMZ | DSM 17845 | 21 Dec. 2005 | Anidral Srl |
| 48 | Lactobacillus fermentum | LF06 | DSMZ | DSM 18295 | 24 May 2006 | Anidral Srl |
| 49 | Lactobacillus fermentum | LF07 | DSMZ | DSM 18296 | 24 May 2006 | Anidral Srl |
| 50 | Lactobacillus fermentum | LF08 | DSMZ | DSM 18297 | 24 May 2006 | Anidral Srl |
| 51 | Lactobacillus fermentum | LF09 | DSMZ | DSM 18298 | 24 May 2006 | Anidral Srl |
| 52 | Lactobacillus gasseri | LGS01 | DSMZ | DSM 18299 | 24 May 2006 | Anidral Srl |
| 53 | Lactobacillus gasseri | LGS02 | DSMZ | DSM 18300 | 24 May 2006 | Anidral Srl |
| 54 | Lactobacillus gasseri | LGS03 | DSMZ | DSM 18301 | 24 May 2006 | Anidral Srl |
| 55 | Lactobacillus gasseri | LGS04 | DSMZ | DSM 18302 | 24 May 2006 | Anidral Srl |
| 56 = | Bifidobacterium adolescentis EI-3 | BA 03 | DSMZ | DSM 18350 | 15 Jun. 2006 | Anidral Srl |
| 32 | Bifidobacterium catenulatum | | | | | |
| | sp./pseudocatenulatum EI-3I, | | | | | |
| | ID 09-255 | | | | | |
| 57 = | Bifidobacterium adolescentis EI-15 | BA 02 | DSMZ | DSM 18351 | 15 Jun. 2006 | Anidral Srl |
| 43 | | | | | | |
| 58 | Bifidobacterium adolescentis EI-18 | BA 05 | DSMZ | DSM 18352 | 15 Jun. 2006 | Anidral Srl |
| | Bifidobacterium animalis subsp. | | | | | |
| | lactis EI-18, ID 09-256 | | | | | |
| 59 | Bifidobacterium catenulatum EI-20 | BC 01 | DSMZ | DSM 18353 | 15 Jun. 2006 | Anidral Srl |
| 60 | Streptococcus thermophilus FRai | MO1 | DSMZ | DSM 18613 | 13 Sep. 2006 | Mofin Srl |
| 61 | Streptococcus thermophilus LB2bi | MO2 | DSMZ | DSM 18614 | 13 Sep. 2006 | Mofin Srl |
| 62 | Streptococcus thermophilus LRci | MO3 | DSMZ | DSM 18615 | 13 Sep. 2006 | Mofin Srl |
| 63 | Streptococcus thermophilus FP4 | MO4 | DSMZ | DSM 18616 | 13 Sep. 2006 | Mofin Srl |
| 64 | Streptococcus thermophilus ZZ5F8 | MO5 | DSMZ | DSM 18617 | 13 Sep. 2006 | Mofin Srl |
| 65 | Streptococcus thermophilus TEO4 | MO6 | DSMZ | DSM 18618 | 13 Sep. 2006 | Mofin Srl |
| 66 | Streptococcus thermophilus S1ci | MO7 | DSMZ | DSM 18619 | 13 Sep. 2006 | Mofin Srl |
| 67 | Streptococcus thermophilus 641bi | MO8 | DSMZ | DSM 18620 | 13 Sep. 2006 | Mofin Srl |
| 68 | Streptococcus thermophilus 277A/1ai | MO9 | DSMZ | DSM 18621 | 13 Sep. 2006 | Mofin Srl |
| 69 | Streptococcus thermophilus 277A/2ai | MO10 | DSMZ | DSM 18622 | 13 Sep. 2006 | Mofin Srl |
| 70 | Streptococcus thermophilus IDC11 | MO11 | DSMZ | DSM 18623 | 13 Sep. 2006 | Mofin Srl |
| 71 | Streptococcus thermophilus ML3di | MO14 | DSMZ | DSM 18624 | 13 Sep. 2006 | Mofin Srl |
| 72 | Streptococcus thermophilus TEO3 | MO15 | DSMZ | DSM 18625 | 13 Sep. 2006 | Mofin Srl |
| 73 | Streptococcus thermophilus G62 | GG1 | DSMZ | DSM 19057 | 21 Feb. 2007 | Mofin Srl |
| 74 | Streptococcus thermophilus G1192 | GG2 | DSMZ | DSM 19058 | 21 Feb. 2007 | Mofin Srl |
| 75 | Streptococcus thermophilus GB18 | GG3 | DSMZ | DSM 19059 | 21 Feb. 2007 | Mofin Srl |
| | | MO2 | | | | |
| 76 | Streptococcus thermophilus CCR21 | GG4 | DSMZ | DSM 19060 | 21 Feb. 2007 | Mofin Srl |
| 77 | Streptococcus thermophilus G92 | GG5 | DSMZ | DSM 19061 | 21 Feb. 2007 | Mofin Srl |
| 78 | Streptococcus thermophilus G69 | GG6 | DSMZ | DSM 19062 | 21 Feb. 2007 | Mofin Srl |
| 79 | Streptococcus thermophilus | YO 10 | DSMZ | DSM 19063 | 21 Feb. 2007 | Anidral Srl |
| 80 | Streptococcus thermophilus | YO 11 | DSMZ | DSM 19064 | 21 Feb. 2007 | Anidral Srl |
| 81 | Streptococcus thermophilus | YO 12 | DSMZ | DSM 19065 | 21 Feb. 2007 | Anidral Srl |
| 82 | Streptococcus thermophilus | YO 13 | DSMZ | DSM 19066 | 21 Feb. 2007 | Anidral Srl |
| 83 | Weissella ssp. WSP 01 | EX | DSMZ | DSM 19067 | 21 Feb. 2007 | Anidral Srl |
| 84 | Weissella ssp. WSP 02 | EX | DSMZ | DSM 19068 | 21 Feb. 2007 | Anidral Srl |
| 85 | Lactobacillus ssp. WSP 03 | EX | DSMZ | DSM 19069 | 21 Feb. 2007 | Anidral Srl |
| 86 | Lactobacillus plantarum LP 09 | OY | DSMZ | DSM 19070 | 21 Feb. 2007 | Anidral Srl |
| 87 | Lactobacillus plantarum LP 10 | OY | DSMZ | DSM 19071 | 21 Feb. 2007 | Anidral Srl |
| 88 | Lactococcus lactis | NS 01 | DSMZ | DSM 19072 | 21 Feb. 2007 | Anidral Srl |
| 89 | Lactobacillus fermentum | LF 10 | DSMZ | DSM 19187 | 20 Mar. 2007 | Anidral Srl |
| 90 | Lactobacillus fermentum | LF 11 | DSMZ | DSM 19188 | 20 Mar. 2007 | Anidral Srl |
| 91 | Lactobacillus casei ssp. | LR05 | DSMZ | DSM 19739 | 27 Sep. 2007 | Anidral Srl |
| | rhamnosus | | | | | |
| 92 | Bifidobacterium bifidum | BB01 | DSMZ | DSM 19818 | 30 Oct. 2007 | Anidral Srl |
| 93 | Lactobacillus delbrueckii subsp. | Lb | DSMZ | DSM 19948 | 28 Nov. 2007 | Anidral Srl |
| | bulgaricus LD 01 | | | | | |
| 94 | Lactobacillus delbrueckii subsp. | Lb | DSMZ | DSM 19949 | 28 Nov. 2007 | Anidral Srl |
| | bulgaricus LD 02 | | | | | |
| 95 | Lactobacillus delbrueckii subsp. | Lb | DSMZ | DSM 19950 | 28 Nov. 2007 | Anidral Srl |
| | bulgaricus LD 03 | | | | | |
| 96 | Lactobacillus delbrueckii subsp. | Lb | DSMZ | DSM 19951 | 28 Nov. 2007 | Anidral Srl |
| | bulgaricus LD 04 | | | | | |
| 97 | Lactobacillus delbrueckii subsp. | Lb | DSMZ | DSM 19952 | 28 Nov. 2007 | Anidral Srl |
| | bulgaricus LD 05 | | | | | |
| 98 | Bifidobacterium pseudocatenulatum | B660 | DSMZ | DSM 21444 | 13 May 2008 | Probiotical SpA |
| 99 | Lactobacillus acidophilus | LA02 | DSMZ | DSM 21717 | 6 Aug. 2008 | Probiotical SpA |
| 100 | Lactobacillus paracasei | LPC 08 | DSMZ | DSM 21718 | 6 Aug. 2008 | Probiotical SpA |
| 101 | Lactobacillus pentosus | LPS 01 | DSMZ | DSM 21980 | 14 Nov. 2008 | Probiotical SpA |
| 102 | Lactobacillus rahmnosus | LR 06 | DSMZ | DSM 21981 | 14 Nov. 2008 | Probiotical SpA |
| 103 | Lactobacillus delbrueckii ssp. | DSMZ | DSMZ | DSM 22106 | 10 Dec. 2008 | Probiotical SpA |
| | delbrueckii | 20074 | | | | |
| 104 | Lactobacillus plantarum | LP1 | DSMZ | DSM 22107 | 10 Dec. 2008 | Probiotical SpA |
| 105 | Lactobacillus salivarius | LS01 | DSMZ | DSM 22775 | 23 Jul. 2009 | Probiotical SpA |
| 106 | Lactobacillus salivarius | LS03 | DSMZ | DSM 22776 | 23 Jul. 2009 | Probiotical SpA |
| 107 | Bifidobacterium bifidum | BB01 | DSMZ | DSM 22892 | 28 Aug. 2009 | Probiotical SpA |
| 108 | Bifidobacterium bifidum | | DSMZ | DSM 22893 | 28 Aug. 2009 | Probiotical SpA |
| 109 | Bifidobacterium bifidum | BB03 | DSMZ | DSM 22894 | 28 Aug. 2009 | Probiotical SpA |
| 110 | Bifidobacterium lactis | BS05 | DSMZ | DSM 23032 | 13 Oct. 2009 | Probiotical SpA |
| 111 | Lactobacillus acidophilus | LA 06 | DSMZ | DSM 23033 | 13 Oct. 2009 | Probiotical SpA |
| 112 | Lactobacillus brevis | LBR01 | DSMZ | DSM 23034 | 13 Oct. 2009 | Probiotical SpA |
| 113 | Bifidobacterium animalis ssp. lactis | BS06 | DSMZ | DSM 23224 | 12 Jan. 2010 | Probiotical SpA |
| 114 | Bifidobacterium longum | BL04 | DSMZ | DSM 23233 | 12 Jan. 2010 | Probiotical SpA |
| 115 | Bifidobacterium longum | BL05 | DSMZ | DSM 23234 | 12 Jan. 2010 | Probiotical SpA |
| 116 | Bifidobacterium bifidum | MB 109 | DSMZ | DSM 23731 | 29 Jun. 2010 | Probiotical SpA |
| 117 | Bifidobacterium breve | MB 113 | DSMZ | DSM 23732 | 29 Jun. 2010 | Probiotical SpA |
| 118 | Bifidobacterium lactis | MB 2409 | DSMZ | DSM 23733 | 29 Jun. 2010 | Probiotical SpA |
| 119 | Lactobacillus reuteri | LRE01 | DSMZ | DSM 23877 | 5 Aug. 2010 | Probiotical SpA |
| 120 | Lactobacillus reuteri | LRE02 | DSMZ | DSM 23878 | 5 Aug. 2010 | Probiotical SpA |
| 121 | Lactobacillus reuteri | LRE03 | DSMZ | DSM 23879 | 5 Aug. 2010 | Probiotical SpA |
| 122 | Lactobacillus reuteri | LRE04 | DSMZ | DSM 23880 | 5 Aug. 2010 | Probiotical SpA |
| 123 | Lactobacillus paracasei ssp. paracasei | LPC09 | DSMZ | DSM 24243 | 23 Nov. 2010 | Probiotical SpA |
| 124 | Lactobacillus acidophilus | LA 07 | DSMZ | DSM 24303 | 23 Nov. 2010 | Probiotical SpA |
| 125 | Bifidobacterium bifidum | BB04 | DSMZ | DSM 24437 | 4 Jan. 2011 | Probiotical SpA |
| 126 | Lactobacillus crispatus | CRL 1251 | DSMZ | DSM 24438 | 4 Jan. 2011 | Probiotical SpA |
| 127 | Lactobacillus crispatus | CRL 1266 | DSMZ | DSM 24439 | 4 Jan. 2011 | Probiotical SpA |
| 128 | Lactobacillus paracasei | CRL 1289 | DSMZ | DSM 24440 | 4 Jan. 2011 | Probiotical SpA |
| 129 | Lactobacillus salivarius | CRL 1328 | DSMZ | DSM 24441 | 4 Jan. 2011 | Probiotical SpA |
| 130 | Lactobacillus gasseri | CRL 1259 | DSMZ | DSM 24512 | 25 Jan. 2011 | Probiotical SpA |
| 131 | Lactobacillus acidophilus | CRL 1294 | DSMZ | DSM 24513 | 25 Jan. 2011 | Probiotical SpA |
| 132 | Lactobacillus salivarius | LS04 | DSMZ | DSM 24618 | 2 Mar. 2011 | Probiotical SpA |
| 133 | Lactobacillus crispatus | LCR01 | DSMZ | DSM 24619 | 2 Mar. 2011 | Probiotical SpA |
| 134 | Lactobacillus crispatus | LCR02 | DSMZ | DSM 24620 | 2 Mar. 2011 | Probiotical SpA |
| 135 | Lacotbacillus acidophilus | LA09 | DSMZ | DSM 24621 | 2 Mar. 2011 | Probiotical SpA |
| 136 | Lactobacillus gasseri | LGS05 | DSMZ | DSM 24622 | 2 Mar. 2011 | Probiotical SpA |
| 137 | Lactobacillus paracasei | LPC11 | DSMZ | DSM 24623 | 2 Mar. 2011 | Probiotical SpA |
| 138 | Bifidobacterium infantis | BI 02 | DSMZ | DSM 24687 | 29 Mar. 2011 | Probiotical SpA |
| 139 | Bifidobacterium bifidum | BB 06 | DSMZ | DSM 24688 | 29 Mar. 2011 | Probiotical SpA |
| 140 | Bifidobacterium longum | BL 06 | DSMZ | DSM 24689 | 29 Mar. 2011 | Probiotical SpA |
| 141 | Bifidobacterium lactis | BS 07 | DSMZ | DSM 24690 | 29 Mar. 2011 | Probiotical SpA |
| 142 | Bifidobacterium longum | PCB133 | DSMZ | DSM 24691 | 29 Mar. 2011 | Probiotical SpA |
| 143 | Bifidobacterium breve | B632 | DSMZ | DSM 24706 | 7 Apr. 2011 | Probiotical SpA |
| 144 | Bifidobacterium breve | B2274 | DSMZ | DSM 24707 | 7 Apr. 2011 | Probiotical SpA |
| 145 | Bifidobacterium breve | B7840 | DSMZ | DSM 24708 | 7 Apr. 2011 | Probiotical SpA |
| 146 | Bifidobacterium longum | B1975 | DSMZ | DSM 24709 | 7 Apr. 2011 | Probiotical SpA |
| 147 | Lactobacillus salivarius | DLV1 | DSMZ | DSM 25138 | 2 Sep. 2011 | Probiotical SpA |
| 148 | Lactobacillus reuteri | LRE05 | DSMZ | DSM 25139 | 2 Sep. 2011 | Probiotical SpA |
| 149 | Lactobacillus reuteri | LRE06 | DSMZ | DSM 25140 | 2 Sep. 2011 | Probiotical SpA |
| 150 | Lactobacillus reuteri | RC 14 | DSMZ | DSM 25141 | 2 Sep. 2011 | Probiotical SpA |
| 151 | Streptococcus thermophilus | ST 10 | DSMZ | DSM 25246 | 19 Sep. 2011 | Probiotical SpA |
| 152 | Streptococcus thermophilus | ST 11 | DSMZ | DSM 25247 | 19 Sep. 2011 | Probiotical SpA |
| 153 | Streptococcus thermophilus | ST 12 | DSMZ | DSM 25282 | 20 Oct. 2011 | Probiotical SpA |
| 154 | Lactobacillus salivarius | DLV8 | DSMZ | DSM 25545 | 12 Jan. 2012 | Probiotical SpA |
| 155 | Bifidobacterium longum | DLBL 07 | DSMZ | DSM 25669 | 16 Feb. 2012 | Probiotical SpA |
| 156 | Bifidobacterium longum | DLBL 08 | DSMZ | DSM 25670 | 16 Feb. 2012 | Probiotical SpA |
| 157 | Bifidobacterium longum | DLBL 09 | DSMZ | DSM 25671 | 16 Feb. 2012 | Probiotical SpA |
| 158 | Bifidobacterium longum | DLBL 10 | DSMZ | DSM 25672 | 16 Feb. 2012 | Probiotical SpA |
| 159 | Bifidobacterium longum | DLBL 11 | DSMZ | DSM 25673 | 16 Feb. 2012 | Probiotical SpA |
| 160 | Bifidobacterium longum | DLBL 12 | DSMZ | DSM 25674 | 16 Feb. 2012 | Probiotical SpA |
| 161 | Bifidobacterium longum | DLBL13 | DSMZ | DSM 25675 | 16 Feb. 2012 | Probiotical SpA |
| 162 | Bifidobacterium longum | DLBL 14 | DSMZ | DSM 25676 | 16 Feb. 2012 | Probiotical SpA |
| 163 | Bifidobacterium longum | DLBL 15 | DSMZ | DSM 25677 | 16 Feb. 2012 | Probiotical SpA |
| 164 | Bifidobacterium longum | DLBL 16 | DSMZ | DSM 25678 | 16 Feb. 2012 | Probiotical SpA |
| 165 | Bifidobacterium longum | DLBL 17 | DSMZ | DSM 25679 | 16 Feb. 2012 | Probiotical SpA |
| 166 | Lactobacillus johnsonii | DLLJO 01 | DSMZ | DSM 25680 | 16 Feb. 2012 | Probiotical SpA |
| 167 | Lactobacillus rhamnosus | DLLR 07 | DSMZ | DSM 25681 | 16 Feb. 2012 | Probiotical SpA |
| 168 | Lactobacillus rhamnosus | DLLR 08 | DSMZ | DSM 25682 | 16 Feb. 2012 | Probiotical SpA |
| 169 | Lactobacillus reuteri | DLLRE 07 | DSMZ | DSM 25683 | 16 Feb. 2012 | Probiotical SpA |
| 170 | Lactobacillus reuteri | DLLRE 08 | DSMZ | DSM 25684 | 16 Feb. 2012 | Probiotical SpA |
| 171 | Lactobacillus reuteri | DLLRE 09 | DSMZ | DSM 25685 | 16 Feb. 2012 | Probiotical SpA |
| 172 | Bifidobacterium longum | DLBL 18 | DSMZ | DSM 25708 | 24 Feb. 2012 | Probiotical SpA |
| 173 | Bifidobacterium infantis | BI 03 | DSMZ | DSM 25709 | 24 Feb. 2012 | Probiotical SpA |
| 174 | Lactobacillus plantarum | LP 09 | DSMZ | DSM 25710 | 24 Feb. 2012 | Probiotical SpA |
| 175 | Bifidobacterium longum | DLBL 19 | DSMZ | DSM 25717 | 1 Mar. 2012 | Probiotical SpA |
| 176 | Bifidobacterium longum | DLBL 20 | DSMZ | DSM 25718 | 1 Mar. 2012 | Probiotical SpA |
| 177 | Lactobacillus salivarius | LS 05 | DSMZ | DSM 26036 | 6 Jun. 2012 | Probiotical SpA |
| 178 | Lactobacillus salivarius | LS 06 | DSMZ | DSM 26037 | 6 Jun. 2012 | Probiotical SpA |
| 179 | Lactobacillus pentosus | LPS 02 | DSMZ | DSM 26038 | 6 Jun. 2012 | Probiotical SpA |
| 180 | Bifidobacterium pseudolongum | BPS 01 | DSMZ | DSM 26456 | 2 Oct. 2012 | Probiotical SpA |
| | ssp. globosum | | | | | |
| 181 | Lactobacillus fermentum | LF15 | DSMZ | DSM 26955 | 1 Mar. 2013 | Probiotical SpA |
| 182 | Lactobacillus fermentum | LF16 | DSMZ | DSM 26956 | 1 Mar. 2013 | Probiotical SpA |
| 183 | Lactobacillus casei | LC03 | DSMZ | DSM 27537 | 24 Jul. 2013 | Probiotical SpA |
| 184 | Lactobacillus crispatus | LCR03 | DSMZ | DSM 27538 | 24 Jul. 2013 | Probiotical SpA |
| 185 | Lactobacillus jensenii | LJE01 | DSMZ | DSM 27539 | 24 Jul. 2013 | Probiotical SpA |
| 186 | Lactobacillus helveticus ID 922 | LH01 | DSMZ | DSM 28153 | 4 Dec. 2013 | Probioiical SpA |
| 187 | Lactobacillus helveticus ID 923 | LH02 | DSMZ | DSM 28154 | 4 Dec. 2013 | Probiotical SpA |
| 188 | Lactococcus lactis ssp. cremoris | LLC02 | DSMZ | DSM 28155 | 4 Dec. 2013 | Probiotical SpA |
| | ID 1612 | | | | | |
| 189 | Lactococcus lactis ssp .cremoris | LLC03 | DSMZ | DSM 28156 | 4 Dec. 2013 | Probiotical SpA |
| | ID 1252 | | | | | |
| 190 | Lactococcus lactis ssp. Lactis | LLL01 | DSMZ | DSM 28157 | 4 Dec. 2013 | Probiotical SpA |
| | ID 1254 | | | | | |
| 191 | Bifidobacterium longum | BL 01 | DSMZ | DSM 28173 | 11 Dec. 2013 | Probiotical SpA |
| 192 | Bifidobacterium longum | BL 02 | DSMZ | DSM 28174 | 11 Dec. 2013 | Probiotical SpA |
| 193 | Bifidobaterium animalis ssp. | Bb1 | DSMZ | DSM 17850 | 23 Dec. 2005 | BioMan Srl |
| | lactis | | | | | |
| 194 | Streptococcus thermophilus | ST 16 BM | DSMZ | DSM 19526 | 13 Jul. 2007 | BioMan Srl |
| 195 | Bifidobacterium infantis | BI 04 | DSMZ | DSM 28651 | 8 Apr. 2014 | Probiotical SpA |
| 196 | Bifidobacterium infantis | BI 05 | DSMZ | DSM 28652 | 8 Apr. 2014 | Probiotical SpA |
| 197 | Streptococcus thermophilus | ST 15 | DSMZ | DSM 28911 | 11 Jun. 2014 | Probiotical SpA |
| 198 | Streptococcus thermophilus | ST 16 | DSMZ | DSM 28912 | 11 Jun. 2014 | Probiotical SpA |
| 199 | Streptococcus thermophilus | ST 17 | DSMZ | DSM 28913 | 11 Jun. 2014 | Probiotical SpA |
| 200 | Lactobacillus fermentum | LF18 | DSMZ | DSM 29197 | 30 Jul. 2014 | Probiotical SpA |
| 201 | Lactobacillus fermentum | LF19 | DSMZ | DSM 29198 | 30 Jul. 2014 | Probiotical SpA |
| 202 | Leuconostoc sp. | LM01 | DSMZ | DSM 29372 | 10 Sep. 2014 | Mofin Srl |
| 203 | Leuconostoc sp. | LM10 | DSMZ | DSM 29373 | 10 Sep. 2014 | Mofin Srl |
| 204 | Leuconostoc sp. | LM11 | DSMZ | DSM 29374 | 10 Sep. 2014 | Mofin Srl |
| 205 | Leuconostoc sp. | LM12 | DSMZ | DSM 29375 | 10 Sep. 2014 | Mofin Srl |
| 206 | Lactobacillusplantarum | LP10 | DSMZ | DSM 29389 | 10 Sep. 2014 | Mofin Srl |
| 207 | Lactobacillusplantarum | LP11 | DSMZ | DSM 29390 | 10 Sep. 2014 | Mofin Srl |
| 208 | Lactobacillusplantarum | LP12 | DSMZ | DSM 29400 | 10 Sep. 2014 | Mofin Srl |
| 209 | Lactobacillusplantarum | LP13 | DSMZ | DSM 29401 | 10 Sep. 2014 | Mofin Srl |
| 210 | Lactobacillus pentosus | LPS03 | DSMZ | DSM 29402 | 10 Sep. 2014 | Mofin Srl |
| 211 | Lactobacillus reuteri | LRE10 | DSMZ | DSM 29403 | 10 Sep. 2014 | Mofin Srl |
| 212 | Lactobacillus brevis | LBR02 | DSMZ | DSM 29404 | 10 Sep. 2014 | Mofin Srl |
| 213 | Lactobacillus salivarius | LS07 | DSMZ | DSM 29476 | 9 Oct. 2014 | Probiotical SpA |
| 214 | Bifidobacterium breve | BR05 | DSMZ | DSM 29494 | 9 Oct. 2014 | Probiotical SpA |
| 215 | Lactococcus lactis ssp. | LCC02 | DSMZ | DSM 29536 | 22 Oct. 2014 | Probiotical SpA |
| | cremoris | | | | | |

The Applicant tested the strains of Table A In order to analyze the molecules characterizing the single cell subpopulations and determine the cytokine assay by E.L.I.S.A. The methods being used are disclosed below.

Analysis of Molecules Characterizing the Single Cell Subpopulations

For the immunophenotypic characterization, samples of 0.1×106 PBMC/100 μl of 1% BSA-PBS are incubated for 30 minutes in the dark with different combinations of monoclonal antibodies (mAb) conjugated with fluorescein isothiocyanate (FITC), phycoerythrin (PE) or peridinin chlorophyll protein (PerCP). The proper isotype controls are also included in the determination. In Table B below the antibody combinations being used are detailed.

TABLE B

| | | mAb | |
|---|---|---|---|
| Immunity | Cell subpopulation | (FL1/FL2/FL3) | Cell selection |
| Natural or | Monocytes | CD14/−/− | CD14+ |
| [symbol] innate | Total Dendritic Cells | Lineage/−/HLA-DR | Lin−/HLA-DR+ |
| | Natural Killer Cells | CD56/−/CD16 | CD56+/CD16+ |
| Specific or acquired | T helper Lymphocytes | CD4/CD8/CD3 | CD3+/CD4+ |
| | Cytotoxic T Lymphocytes | | CD3+/CD8+ |
| | Regulatory T | CD25/−/CD3 | CD3+/CD25+ |
| | Lymphocytes | | |
| | Total B Lymphocytes | CD19/−/CD20 | CD19+/CD20+ |

Lineage = CD3, CD14, CD16, CD19, CD20, CD56 [symbol]
indicates data missing or illegible when filed

Upon incubation, the samples are washed with 1.5 mL 1% BSA-PBS for removing any trace of excess antibodies (centrifugation 1600 rpm for 5 minutes). Cells are fixed by adding 200 μl of 1% PFA-PBS and stored at 4° C. Within 24 hours after preparation, the samples are analyzed by a cytofluorometer FACScalibur, selecting the cells so that to exclude contaminant cellular debris from the analysis.

Cytokine Assay with E.L.I.S.A.

The present method allows to determining the concentration of human interleukins present in cell culture supernatants, serum, plasma and urine by Enzyme-Linked Immunosorbent Assays (E.L.I.S.A.). As regards the cytokine assay, in the present method, the KIT Human ELISA Ready-SET-Go® from eBioscience Company are used.

Principle of the Method

The Enzyme-Linked ImmunoSorbent Assay (E.L.I.S.A.), is an immunoenzymatic test aimed to verify the presence of a specific antigen (Ag) in a given sample. In particular, the E.L.I.S.A. method, to which the present method is related, is as direct or sandwich type. A monoclonal antibody (capture Ab), able to specifically detect the cytokine of interest, is used for coating the wells of a microplate. The samples are distributed in different microwells so that al the cytokine of interest being present can bind to the immobilized Ab. Thus, a biotin-conjugated antibody (detection Ab), able to detect and bind the cytokine of interest, and the enzyme peroxidase (HRP), conjugated to Streptavidin, being able to binding biotin and anchoring the enzyme itself to the complex are thus sequentially added. Finally, the colorless chromogenic substrate is added, which in the presence of the enzyme is oxidized, thus developing a blue color, with an intensity proportional to the amount of immobilized cytokine. The reaction is stopped by adding sulfuric acid, which leads to a color change from blue to yellow.

Procedure

It has to be noted that al the samples and reagents to be used for performing the method should be brought to room temperature before their use. All the described steps should be performed at room temperature.

Take a number of 96 flat-bottom well plates, specific for E.L.I.S.A. assays (Corming Costar 9018 ELISA, included in the kit), so that to have a well for each sample to be tested and an adequate number of wells for preparing the standard calibration curve (16 wells in total).

Dilute the stock solution of primary antibody (Capture Ab; final concentration 10 μg/ml) 250 times in the coating solution 1× (stock solution 10×, included in the kit, to be diluted 1:10 in sterile distilled water).

Distribute 100 μl of such a solution in all the wells; cover the plate with adhesive strip, and incubate overnight (O.N.) at 4° C. (refrigerator).

Wash 5 times each well with 200 μl of washing solution (0.05% Tween 20 in PBS). By means of a vacuum pump, suck the liquid from the last washing in all the wells and turn over the plate onto an absorbent paper sheet for removing any residue of the solution.

Add 200 μl of assay buffer 1× (stock solution 5×, included in the Kit, to be diluted 1:5 in sterile distilled water) in all the wells and incubate for 1 hour at room temperature (RT).

Wash 5 times each well with 200 μl of washing solution. By means of a vacuum pump, suck the liquid from the last washing in all the welts and turn over the plate onto an absorbent paper sheet for removing any residue of the solution.

Prepare the solution containing the human recombinant cytokine, required for the standard calibration curve, according to the table below, taking take to centrifuge all the tubes before opening them:

TABLE C

| | 8 point curve | Dilution | STOCK | |
|---|---|---|---|---|
| Cytokine | range (pg/ml) | curve | Ab (stock) | Assay Buffer 1× |
| IL-4 | 2-200 | 1:2 | 4 μl | 20 ml |
| IL-12p70 | 4-500 | 1:2 | 10 μl | 20 ml |
| IFN-g | 4-500 | 1:2 | 10 μl | 20 ml |
| IL-17A | 4-500 | 1:2 | 5 μl | 10 ml |

As from the scheme below, distribute 100 μl of assay buffer 1× in wells from C2 to C8. Distribute 200 μl of STOCK (prepared according to the above table) in well C1 and proceed with serial dilutions 1:2 as specified in the scheme. The dilutions can be performed directly in the wells or in Eppendorf tubes.

The curve should be prepared in duplicate.

Add 100 μl of each sample to be tested (S, in duplicate) in the given well according to the scheme of the plate. Cover the plate with adhesive strip and incubate overnight (O.N.) at 4° C. (refrigerator).

Prepare the solution containing the secondary biotinylated antibody (detection Ab), by diluting 250 times the stock solution in Assay buffer 1×, as set forth following the manufacturer's instructions.

Wash 5 times each well with 200 μl of washing solution. By means of a vacuum pump, suck the liquid from the last washing in all the wells and turn over the plate onto an absorbent paper sheet for removing any residue of the solution.

Distribute 100 μl of the solution containing the secondary Ab in all the wells, cover the plate with the adhesive strip, and incubate for 1 hour at RT.

Prepare the solution containing the enzyme Avidin-HRP, by diluting 250 times the stock solution in Assay buffer 1×, as set forth in the manufacturer's Instructions.

Wash 5 times each well with 200 μl of washing solution. By means of a vacuum pump, suck the liquid from the last washing in al the wells and turn over the plate onto an absorbent paper sheet for removing any residue of the solution.

Distribute 100 μl of the solution with the enzyme in all the wells, cover the plate with the adhesive strip and incubate for 30 min at RT.

Wash 7 times each well with 200 μl of washing solution. By means of a vacuum pump, suck the liquid from the last washing in all the wells and turn over the plate onto an absorbent paper sheet for removing any residue of the solution.

Add 100 μl of Substrate (1×TMB solution) in all the wells and incubate at RT for 10 minutes in the dark (cover the plate with aluminium foil paper).

Stop the reaction by adding to al the wells 50 μl of 2N sulfuric acid (the color will change from blue to yellow).

Read the plate with a spectrophotometric reader for 96-well plates within 30 min. from the development at a 450 nm wavelength.

Calculation/Expression of the Results

By using a calculator Excell sheet or any software provided in the reader used, insert all the absorbance values read by the spectrophotometer. Calculate the average of the absorbance values for each duplicate (standard and samples).

For the standard curve only, match with the absorbance values the known concentrations of recombinant cytokine of the different dilutions.

By using the standard curve values, plot a graph with the average absorbance values on the x-axis and the known concentrations of cytokine on the y-axis.

Plot the standard calibration curve, which better fits the points of the graph (R=1 is the perfect one). The construction of a 5-parameter curve (five parameter logistic 5-PL curve-fit) is recommended.

Insert in the graph the equation of the plotted curve and the R value.

Then determine the cytokine concentration in every single sample by extrapolating the equation of the plotted standard curve (see the following example).

Example: Table of absorbance values vs concentrations for the construction of the standard calibration curve, being obtained following to the IL-4 cytokine assay.

TABLE D

| X | Y |
|---|---|
| Average OD | pg/ml |
| 1.964 | 500 |
| 1.006 | 350 |
| 0.539 | 125 |
| 0.278 | 62.5 |
| 0.155 | 31.3 |
| 0.099 | 15.6 |
| 0.052 | 7.8 |
| 0.022 | 0 |

For each sample, by replacing the X value of the curve equation with the OD being read, it is possible to obtain, by extrapolation, the unknown concentration of IL-4 therein present.

Interpretation of the Results

When the procedure is accurately performed, the amounts of the tested cytokine in each tested sample should be comprised within the range of the standard calibration curve values. Cytokine concentrations outside the standard calibration curve should be considered as Inaccurate. Especially for greater values it is recommended to further dilute the tested sample by using Assay Buffer.

Results are set forth in Table E.

With reference to the composition of the 5 strains: Bifidobacterium longum DLBL 07 DSM 25669, Bifidobacterium longum DLBL 08 DSM 25670, Bifidobacterium longum DLBL 09 DSM 25671, Bifidobacterium longum DLBL 10 DSM 25672 and Bifidobacterium longum DLBL 11 DSM 25673, the results are as follows: IL-12p70=1.02±0.02; IL-4=3.19±0.41; IFN-g=16.17±4.84; IL-17A=1.11-0.04. The variation of the Th1/Th2 ratio versus the baseline value is 3.10±1.06.

Tests of indole bioconversion by strains belonging to the species Bifidobacterium longum

The study investigated the ability and capability of a mixture based on the 5 strains of bacteria (briefly, “five strain mixture”) belonging to the species B. longum: Bifidobacterium longum DLBL 07 DSM 25669, Bifidobacterium longum DLBL 08 DSM 25670, Bifidobacterium longum DLBL 09 DSM 25671, Bifidobacterium longum DLBL 10 DSM 25672 and Bifidobacterium longum DLBL 11 DSM 25673, in a weight ratio of 1:1:1:1:1 (10⁹ CFU/g for each bacterial strain), to bioconverting the uremic toxin indole. The bioconversion tests were performed under cell growth conditions.

Materials and Methods

A 1M stock solution of indole in DMSO (Dimethyl sulfoxide) was prepared. A freeze-dried mixture of the five strains of Bifidobacteria as described above was activated in MRS agar (BD Difco, Sparks, USA) by adding L-cysteine HCl and incubated under anaerobic conditions (N₂ 85%, CO₂ 10%, H₂ 5%) at a temperature of 37° C. and for 48 h.

For assessing the indole conversion, the activated culture of Bifidobacteria was thus inoculated (10% v/v) in 10 ml of MRS containing 1 mM indole and incubated under anaerobic conditions (N₂ 85%, CO₂ 10%, H₂ 5%) at a temperature of 37° C. and for 48 h.

After 48 h, aliquots were taken and centrifuged (13.000×g for 5 min at 4° C.), the obtained supernatant was then filtered (liter of cellulose acetate, 0.22 μm). 10 μl of the filtered supernatant were next analyzed by HPLC (Agilent 1100, Agilent Technologies Inc., Santa Clara, Calif., USA) equipped with a detector at a variable wavelength and a column ZORBAX Eclipse XDB-C18 (rapid resolution, 1.8 μm particle size, 4.6×50 mm, Agilent). The mobile phase consisted of 40 mM aqueous ammonium acetate solution (Solvent A) and acetonitrile (Solvent B). The flow was set at 0.8 ml/min. For the HPLC analysis, the following gradient was thus applied: 0-10 minutes linear from 10% to 50% of Solvent B; 10-11 min, linear up to 90%; 11-16 min, isocratic 90%; 16-17 min, linear 10%; 17-20 min, isocratic 10%. The analyte indole was identified at a 275 nm wavelength and quantified by using an external standard.

Results

The mixture of five strains of Bifidobacterium longum as described above was analyzed for its ability to convert the uremic toxin indole, therefore decreasing the concentration thereof in the culture broth.

The experiment was performed under active bacterial growth conditions in the culture broth. In the absence of the mixture of Bifidobacterium longum the indole concentration in the culture broth remains unchanged. In the presence of the activated mixture of Bifidobacterium longum, the indole concentration (1 mM) decreased by 24%±2% after 48 h of incubation under anaerobic conditions (N₂ 85%, CO₂ 10%, H₂ 5%) and at a temperature of 37° C. The presence of the uremic toxin indole in the culture broth did not limit the bacterial growth of the mixture of Bifidobacterium longum (P>0.05).

Embodiments (En) of the present invention are set forth below:

E1. Strain of bacterium belonging to the genus Lactobacillus or Bifidobacteria, said strain being selected from those having:
(i) a capability to modulate the immune system by modulating the production of the anti-inflammatory cytokine, such as IL-4, to a value comprised from 2.5 to 4.5 folds, relative to the baseline value set equal to 1; and
(ii) a capability to modulate the immune system by modulating the production of the proinflammatory cytokine, such as IL-12p70, to a value comprised from 0.85 to 1.05 folds, relative to the baseline value set equal to 1; and
(iii) a capability to modulate the Immune system by modulating the production of the proinflammatory cytokine, such as IFN-gamma, to a value comprised from 7 a 19.5 folds, relative to the baseline value set equal to 1; and
(iv) a capability to modulate the immune system by modulating the production of proinflammatory cytokines, such as IL-17, to a value comprised from 0.90 to 1.40 folds, relative to the baseline value set equal to 1; and
v) an overall capability to modulate the ratio of proinflammatory/anti-inflammatory cytokines to give a value of the Th1/Th2 ratio comprised from 2.90 to 4.50;
for use in modulating the immune system, delaying the aging process, maintaining a long-lasting homeostasis condition.
E2. The strain of bacterium for use according to E1, wherein said strain has:
(i) a capability to modulate the immune system by modulating the production of the anti-inflammatory cytokine, such as IL-4, to a value comprised from 3 to 4 folds, relative to the baseline value set equal to 1; and
(ii) a capability to modulate the immune system by modulating the production of the proinflammatory cytokine, such as IL-12p70, to a value comprised from 0.90 to 1 folds, relative to the baseline value set equal to 1; and
(iii) a capability to modulate the immune system by modulating the production of the proinflammatory cytokine, such as IFN-gamma, to a value comprised from 8 to 18 folds, relative to the baseline value set equal to 1; and
(iv) a capability to modulate the immune system by modulating the production of proinflammatory cytokines, such as IL-17, to a value comprised from 0.95 to 1.30 folds, relative to the baseline value set equal to 1; and
(v) an overall capability to modulate the ratio of proinflammatory/anti-inflammatory cytokines to give a value of the Th1/Th2 ratio comprised from 3 to 4.
E3. The strain of bacterium for use according to E1 or E2, wherein said strain belongs to the species Bifidobacterium longum; preferably said strain is selected from the group comprising or, alternatively, consisting of Bifidobacterium longum DLBL 07 DSM 25669, Bifidobacterium longum DLBL 08 DSM 25670, Bifidobacterium longum DLBL 09 DSM 25671, Bifidobacterium longum DLBL 10 DSM 25672, Bifidobacterium longum DLBL 11 DSM 25673, or mixtures thereof.
E4. Composition for oral use comprising or, alternatively, consisting of:
a) at least a strain of bacteria belonging to the genus Lactobacillus or Bifidobacteria which fulfils all the conditions (i)-(v) as claimed in E1 or E2 or E3; for use in modulating the immune system, delaying the aging process, maintaining a long-lasting homeostasis condition.
E5. The composition for use according to E4, wherein said composition is for use in the treatment of renal failure, preferably acute or chronic, for the maintenance of a long-lasting homeostasis condition.
E6. The composition for use according to E4 or E5, wherein said composition is for use in the reduction of uremic toxins, for the maintenance of a long-lasting homeostasis condition.
E7. The composition for use according to E4 or E5 or E6, wherein said uremic toxins are of bacterial origin, preferably are selected from indole and/or cresol.
E8. The composition for use according to any one of E4-E7, wherein said composition comprises or, alternatively, consists of:
(a) at least a strain of bacteria belonging to the genus Lactobacillus or Bifidobacteria which fulfils all the conditions (i)-(v) as claimed in E1 or E2 or E3; and
(b) a specific mucoadherent gelling complex, consisting of exopolysaccharides (EPS) of bacterial origin produced in situ by the strain of bacterium Streptococcus thermophilus ST10 DSM25246 and a polysaccharide of plant origin; preferably tara gum.
E9. The composition for use according to any one of E4-E8, wherein said composition further comprises the strains Lactobacillus buchneri Lb 26 (DSM 16341) and/or Bifidobacterium lactis Bb1 (DSM 17850) which, respectively, provide selenium and zinc in a form highly assimilable by the body; preferably said strains are in tyndallized form.
E10. The composition for use according to any one of E4-E9, wherein said composition further comprises the strain Bifidobacterium lactis BA05 (DSM 18352) which is able to synthesize folates.
E11. The composition for use according to any one of E4-E10, wherein said composition further comprises some prebiotic fibers and carbohydrates with bifidogenic activity selected from inulin, fructo-oligosaccharides (FOS), galacto- and trans-galacto-oligosaccharides (GOS and TOS), gluco-oligosaccharides (GOSα), xylo-oligosaccharides, (XOS), chitosan-oligosaccharides (COS), soya-oligosaccharides (SOS), isomalto-oligosaccharides (IMOS), resistant starch, pectins, psyllium, arabinogalactans, glucomannans, galactomannans, xylans, lactosucrose, lactulose, lactitol and many other types of gums, preferably tara gum, acacia, locust, oat, bamboo fiber, citrus fruit fibers and, in general, fibers containing a soluble and an Insoluble portion, in a variable ratio from each other.
E12. The composition for use according to any one of E4-E11, wherein said composition has a bacterial load comprised from 1×10⁶ to 1×10¹¹ UFC/g, preferably from 1×10⁸ to 1×10¹⁰ UFC/g.
E13. The composition for use according to any one of E4-E12, wherein said composition contains the strains of bacteria belonging to the genus Lactobacillus or Bifidobacteria which fulfil al the conditions (i)-(v), according to E1 or E2 or E3, in an amount comprised from 0.1 to 65% by weight, preferably from 0.5 to 15% by weight; even more preferably from 1 to 10% by weight, relative to the total weight of the composition.

TABLE E
proinflammatory strains (Th1)

| | | | | | Variation of | | | | |
|---|---|---|---|---|---|---|---|---|---|
| | | | | | Th1/Th2 | | | | |
| Proinflammatory | | | | | ratio vs | [symbol] gle cytokine variation relative to the baseline value | | | |
| strains (Th1) | Abbreviation | ID | Deposit No. | Strain ratio | baseline | IL-12p70 | IL-4 | IFN-g | IL-17A |
| L. casei | LPC 08 | 1696 | DSM21718 | 1.76 ± 0.32 | 4.48 ± 1.01 | 1.83 ± 0.30 | 1.26 ± 0.20 | 7.83 ± 0.56 | 1.00 ± 0.01 |
| subsp. paracasei | | | | | | | | | |
| L. fermentum | LF 11 | 1639 | DSM 19188 | 2.15 ± 0.23 | 2.37 ± 0.43 | 1.10 ± 0.04 | 0.90 ± 0.05 | 8.67 ± 1.05 | 1.16 ± 0.03 |
| L. casei | LC 03 | 1872 | DSM 27537 | 2.34 ± 0.24 | 3.15 ± 0.30 | 3.84 ± 0.53 | 0.99 ± 0.26 | 14.30 ± 2.12 | 0.71 ± 0.15 |
| L. paracasei | LPC 00 | 1076 | LMG P-21380 | 1.76 ± 0.32 | 4.48 ± 1.01 | 1.83 ± 0.31 | 1.26 ± 0.20 | 7.83 ± 0.56 | nv |
| L. paracasei | LPC 00 | 1076 | LMG P-21380 | 1.33 ± 0.13 | 2.10 ± 0.21 | 2.14 ± 0.07 | 3.35 ± 0.28 | 8.06 ± 0.95 | 1.00 ± 0.01 |
| L. plantarum | LP 09 | 1837 | DSM 25710 | 3.60 ± 0.81 | 3.53 ± 1.07 | 4.39 ± 0.46 | 1.32 ± 0.02 | 22.29 ± 4.09 | 2.02 ± 0.00 |
| L. pentosus | LPS 01 | 1702 | DSM 21880 | 4.09 ± 0.29 | 8.96 ± 0.83 | 9.46 ± 1.30 | 0.60 ± 0.24 | 19.54 ± 1.68 | 2.93 ± 0.63 |
| L. reuteri | LRE 01 | 1775 | DSM 23877 | 1.04 ± 0.20 | 2.11 ± 0.47 | 3.90 ± 1.24 | 1.80 ± 0.68 | 2.79 ± 0.61 | 0.86 ± 0.06 |
| L. reuteri | LRE 03 | 1777 | DSM 23879 | 8.34 ± 2.15 | 21.0 ± 8.87 | 6.13 ± 1.25 | 0.41 ± 0.17 | 47.02 ± 4.38 | 1.75 ± 0.14 |
| L. rhamnosus | LR 05 | 1602 | DSM 19739 | 0.70 ± 0.17 | 2.21 ± 0.58 | 1.11 ± 0.08 | 1.32 ± 0.30 | 4.16 ± 1.06 | nv |
| L. salivarius | LS06 (L166) | 1727 | DSM 26037 | 1.19 ± 0.13 | 2.38 ± 0.56 | 5.96 ± 0.69 | 1.30 ± 0.14 | 4.95 ± 0.92 | 0.43 ± 0.06 |
| L. salivarius | DL V8 | 1813 | DSM 25545 | 2.52 ± 0.31 | 2.43 ± 0.42 | 2.24 ± 0.33 | 4.00 ± 1.27 | 2.74 ± 0.57 | 1.06 ± 0.11 |
| B. animalis | BS 01 | 1195 | LMG P-21384 | 0.61 ± 0.05 | 2.36 ± 0.47 | 2.83 ± 0.20 | 1.71 ± 0.29 | 6.84 ± 0.81 | 2.44 ± 0.52 |
| subsp lactis | | | | | | | | | |
| B. longum | DL BL07 | 1820 | DSM 25669 | 2.14 ± 0.55 | 3.38 ± 0.85 | 0.96 ± 0.01 | 3.12 ± 0.27 | 15.25 ± 4.01 | 1.01 ± 0.01 |
| B. longum | DL BL08 | 1823 | DSM 25670 | 2.04 ± 0.40 | 3.21 ± 0.61 | 0.91 ± 0.02 | 3.44 ± 0.78 | 8.95 ± 1.77 | 1.26 ± 0.09 |
| B. longum | DL BL09 | 1821 | DSM 25671 | 1.72 ± 0.33 | 2.73 ± 0.54 | 0.97 ± 0.02 | 3.31 ± 0.60 | 12.01 ± 2.75 | 1.05 ± 0.03 |
| B. longum | D LBL10 | 1824 | DSM 25672 | 2.32 ± 0.43 | 3.66 ± 0.67 | 0.97 ± 0.01 | 3.73 ± 0.39 | 11.35 ± 2.09 | 1.11 ± 0.07 |
| B. longum | DL BL11 | 1825 | DSM 25673 | 1.39 ± 0.43 | 2.21 ± 0.70 | 0.97 ± 0.01 | 3.42 ± 0.37 | 11.85 ± 3.78 | 1.01 ± 0.01 |
| B. longum | BL01 | 1293 | DSM 28173 | 2.71 ± 0.42 | 6.28 ± 1.72 | 3.18 ± 0.23 | 1.03 ± 0.01 | 21.9 ± 4.67 | 0.74 ± 0.18 |
| B. longum | BL02 | 1295 | DSM 28174 | 13.6 ± 3.04 | 6.46 ± 0.96 | 2.85 ± 1.14 | 0.97 ± 0.04 | 20.84 ± 0.89 | 1.69 ± 0.36 |
| B. longum | BL03 | 1152 | DSM 16603 | 4.48 ± 1.25 | 9.6 ± 2.36 | 2.47 ± 0.64 | 1.08 ± 0.04 | 24.44 ± 5.45 | 0.82 ± 0.13 |
| B. longum | BL04 | 1740 | DSM23233 | 1.93 ± 0.30 | 4.82 ± 1.87 | 2.45 ± 0.60 | 1.03 ± 0.05 | 19.11 ± 5.38 | 1.22 ± 0.18 |
| B. longum | W11 | 1114 | no deposit | 2.84 ± 0.43 | 5.23 ± 1.2 | 1.39 ± 0.10 | 3.23 ± 0.49 | 26.01 ± 7.40 | 1.00 ± 0.14 |
| B. longum | W11 wt | 1161 | no deposit | 2.60 ± 0.31 | 4.68 ± 0.79 | 1.01 ± 0.02 | 3.44 ± 0.37 | 27.42 ± 6.78 | 0.83 ± 0.09 |
| B. longum | PCB133 | 1687 | DSM 24691 | 2.17 ± 0.36 | 4.16 ± 1.04 | 2.05 ± 0.13 | 1.42 ± 0.18 | 29.09 ± 8.25 | 1.04 ± 0.16 |
| B. longum | BL05 | 1352 | DSM 23234 | 2.79 ± 0.71 | 5.89 ± 1.38 | 1.82 ± 0.30 | 0.92 ± 0.09 | 14.94 ± 2.28 | 1.19 ± 0.19 |
| B. longum | BL06 | no ID | DSM 24689 | 1.69 ± 0.18 | 4.19 ± 1.04 | 2.05 ± 0.10 | 1.41 ± 0.19 | 31.90 ± 3.96 | 0.98 ± 0.16 |

Proinflammatory strains (Th2)

| | | | | | Variation of | | | | |
|---|---|---|---|---|---|---|---|---|---|
| | | | | | Th1/Th2 | | | | |
| Anti-inflammatory | | | | | ratio vs | Single cytokine variation relative to the baseline | | | |
| strains (Th2) | Abbreviation | ID | Deposit No. | Strain ratio | baseline | IL-12p70 | IL-4 | IFN-g | IL-17A |
| L. acidophilus | LA02 | 1688 | DSM 21717 | 0.35 ± 0.05 | 0.90 ± 0.18 | 6.59 ± 0.43 | 1.26 ± 0.34 | 4.91 ± 0.70 | 0.90 ± 0.06 |
| L. deldrueckii | LDD01 | 1391 | DSM 22106 | 0.54 ± 0.10 | 0.58 ± 0.13 | 1.08 ± 0.02 | 0.84 ± 0.07 | 6.46 ± 0.92 | 0.65 ± 0.03 |
| subsp. delbrueckii | | | | | | | | | |
| L. fermentum | LF09 | 1462 | DSM 18298 | 0.29 ± 0.07 | 0.29 ± 0.05 | 2.27 ± 0.32 | 1.04 ± 0.08 | 0.80 ± 0.15 | 0.86 ± 0.29 |
| L. fermentum | LF10 | 1637 | DSM 19187 | 0.43 ± 0.10 | 0.44 ± 0.09 | 1.09 ± 0.04 | 0.90 ± 0.04 | 4.25 ± 0.4 | 0.85 ± 0.12 |
| L. plantarum | LP01 | 1171 | LMG P-21021 | 0.15 ± 0.04 | 0.30 ± 0.06 | 1.61 ± 0.31 | 8.85 ± 2.64 | 1.77 ± 0.42 | 0.43 ± 0.08 |
| L. plantarum | LP02 | 91 | LMG P-21020 | 0.31 ± 0.04 | 0.62 ± 0.10 | 6.31 ± 1.19 | 9.02 ± 1.24 | 4.59 ± 0.59 | 0.91 ± 0.11 |
| L. reuteri | LRE02 | 1774 | DSM 23878 | 0.21 ± 0.03 | 0.36 ± 0.06 | 1.06 ± 0.02 | 2.91 ± 0.87 | 1.19 ± 0.12 | 1.09 ± 0.10 |
| L. reuteri | LRE04 | 1779 | DSM 23880 | 0.35 ± 0.04 | 0.79 ± 0.20 | 1.69 ± 1.25 | 1.61 ± 0.57 | 1.72 ± 0.39 | 0.67 ± 0.07 |
| L. reuteri | DL LRE07 | ? | DSM 25683 | 0.15 ± 0.01 | 0.25 ± 0.02 | 0.83 ± 0.13 | 1.61 ± 0.57 | 1.00 ± 0.07 | 0.70 ± 0.07 |
| L. reuteri | DL LRE08 | 1841 | DSM 25684 | 0.15 ± 0.02 | 0.25 ± 0.02 | 0.68 ± 0.13 | 7.66 ± 3.17 | 0.93 ± 0.06 | 2.05 ± 0.38 |
| L. reuteri | DL LRE09 | 1842 | DSM 25685 | 0.25 ± 0.02 | 0.56 ± 0.13 | 3.61 ± 0.98 | 1.82 ± 0.82 | 1.22 ± 0.29 | 0.54 ± 0.03 |
| L. rhamnosus | LR06 | 1697 | DSM 21981 | 0.34 ± 0.06 | 0.70 ± 0.06 | 1.11 ± 0.06 | 1.60 ± 0.34 | 2.64 ± 0.83 | 1.91 ± 0.04 |
| L. salivarius | LS01 | 1797 | DSM 22775 | 0.13 ± 0.04 | 0.46 ± 0.13 | 1.95 ± 0.17 | 2.02 ± 0.48 | 1.44 ± 0.13 | 0.7 ± 0.13 |
| L. salivarius | LS04 | ? | DSM 24618 | | | | | | |
| L. salivarius | LS03 | 1382 | DSM 22776 | 0.22 ± 0.06 | 0.64 ± 0.13 | 2.43 ± 0.27 | 1.32 ± 0.11 | 0.72 ± 0.26 | 0.46 ± 0.06 |
| L. salivarius | DLV1 | 1806 | DSM 25138 | 0.67 ± 0.04 | 0.63 ± 0.08 | 1.82 ± 0.18 | 2.16 ± 0.51 | 1.40 ± 0.13 | 0.81 ± 0.13 |
| L. salivarius | LS05 (L66) | 1719 | DSM 26036 | 0.30 ± 0.02 | 0.57 ± 0.11 | 4.74 ± 0.40 | 19.51 ± 1.51 | 1.80 ± 0.09 | 1.18 ± 0.19 |
| L. salivarius | LS02 | 1468 | DSM 20555 | 0.07 ± 0.02 | 0.40 ± 0.23 | 2.41 ± 0.48 | 2.20 ± 0.25 | 1.32 ± 0.67 | 0.62 ± 0.10 |
| B. lactis | BA05 | 1518 | DSM 18352 | 0.15 ± 0.02 | 0.39 ± 0.05 | 1.90 ± 0.29 | 1.18 ± 0.20 | 1.24 ± 0.08 | 1.07 ± 0.07 |
| B. breve | BR03 | 1270 | DSM 16604 | 0.08 ± 0.01 | 0.35 ± 0.05 | 2.30 ± 0.50 | 2.3 ± 0.20 | 2.20 ± 0.20 | 0.74 ± 0.18 |
| B. breve | BR03 | 1270 | DSM 16604 | 0.49 ± 0.04 | 0.93 ± 0.18 | 1.08 ± 0.25 | 1.07 ± 0.02 | 6.92 ± 1.02 | 0.85 ± 0.16 |
| B. pseudolongum | BPS01 | 1812 | DSM 26456 | 0.10 ± 0.02 | 0.22 ± 0.09 | 0.92 ± 0.06 | 1.00 ± 0.02 | 0.64 ± 0.33 | 0.74 ± 0.15 |
| subsp. globosum | | | | | | | | | |
| B. longum | B1975 | 1742 | DSM 24709 | 0.94 ± 0.13 | 0.51 ± 0.13 | 4.13 ± 0.60 | 1.05 ± 0.07 | 2.99 ± 0.71 | 0.85 ± 0.17 |

(*) number of folds the bacterium induces:
N > 1 increase of the tested cytokine
N < 1 decrease of the tested cytokine
N = 1 no variation of the tested cytokine
bold numbers = statistically significant variation [symbol]
indicates data missing or illegible when filed

1. A method for modulating an immune system, delaying an aging process, maintaining a long-lasting homeostasis condition in a subject, the method comprising:
administering to the subject an effective amount of one or more strains of bacterium belonging to the genus Lactobacillus or Bifidobacteria, and being capable to modulate the immune system of the subject by modulating:
production of
an anti-inflammatory cytokine to a value comprised from 2.5 to 4.5 folds, relative to a baseline value set equal to 1,
a first proinflammatory cytokine to a value comprised from 0.85 to 1.05 folds, relative to the baseline value set equal to 1,
a second proinflammatory cytokine to a value comprised from 7 to 19.5 folds, relative to the baseline value set equal to 1, and
a third proinflammatory cytokine to a value comprised from 0.90 to 1.40 folds, relative to the baseline value set equal to 1; and
a ratio of proinflammatory/anti-inflammatory cytokines to provide a Th1/Th2 ratio from 2.90 to 4.50.

2. The method according to claim 1, wherein said one or more strains being capable to modulate the immune system of the subject by modulating:
production of
the first anti-inflammatory cytokine to a value comprised from 3 to 4 folds, relative to the baseline value set equal to 1,
the first proinflammatory cytokine to a value comprised from 0.90 to 1 folds, relative to the baseline value set equal to 1,
the second proinflammatory cytokine to a value comprised from 8 to 18 folds, relative to the baseline value set equal to 1, and
the third proinflammatory cytokine to a value comprised from 0.95 to 1.30 folds, relative to the baseline value set equal to 1; and
the ratio of proinflammatory/anti-inflammatory cytokines to provide a Th1/Th2 ratio from 3 to 4.

3. The method according to claim 1, wherein said one or more strains belong to the species Bifidobacterium longum.

4. A composition for oral use comprising:
at least one strain of bacteria belonging to the genus Lactobacillus or Bifidobacteria capable of modulating the immune system of a subject, delaying an aging process of the subject, maintaining a long-lasting homeostasis condition in the subject, wherein the at least one strain of bacteria is capable to modulate:
production of
an anti-inflammatory cytokine to a value from 2.5 to 4.5 folds, relative to a baseline value set equal to 1,
a first proinflammatory cytokine to a value from 0.85 to 1.05 folds, relative to the baseline value set equal to 1,
a second proinflammatory cytokine to a value from 7 to 19.5 folds, relative to the baseline value set equal to 1, and
a third proinflammatory cytokines to a value from 0.90 to 1.40 folds, relative to the baseline value set equal to 1; and
a ratio of proinflammatory/anti-inflammatory cytokines to provide a Th1/Th2 ratio from 2.90 to 4.50.

5. A method of treating renal failure, for the maintenance of a long-lasting homeostasis condition in a subject, comprising:
administering to the subject an effective amount of the composition of claim 4.

6. A method of reducing uremic toxins, for the maintenance of a long-lasting homeostasis condition in a subject, comprising:
administering to the subject an effective amount of the composition of claim 4.

7. The method of claim 6, wherein said uremic toxins are of bacterial origin.

8. The composition according to claim 4, wherein said composition further comprises:
a specific mucoadherent gelling complex, consisting of exopolysaccharides (EPS) of bacterial origin produced in situ by bacterium Streptococcus thermophilus ST10 DSM25246 and a polysaccharide of plant origin.

9. The composition according to claim 4, wherein said composition further comprises Lactobacillus buchneri Lb 26 (DSM 16341) and/or Bifidobacterium lactis Bb1 (DSM 17850).

10. The composition according to claim 4, wherein said composition further comprises the strain Bifidobacterium lactis BA05 (DSM 18352).

11. The composition according to claim 4, wherein said composition further comprises prebiotic fibers and carbohydrates with bifidogenic activity selected from the group consisting of inulin, fructo-oligosaccharides (FOS), galacto- and trans-galacto-oligosaccharides (GOS and TOS), gluco-oligosaccharides (GOSα), xylo-oligosaccharides, (XOS), chitosan-oligosaccharides (COS), soya-oligosaccharides (SOS), isomalto-oligosaccharides (IMOS), resistant starch, pectins, psyllium, arabinogalactans, glucomannans, galactomannans, xylans, lactosucrose, lactulose, lactitol, tara gum, acacia, locust, oat, bamboo fiber, citrus fruit fibers and fibers containing a soluble and an insoluble portion.

12. The composition according to claim 4, wherein said composition has a bacterial load comprised from 1×10⁶ to 1×10¹¹ UFC/g.

13. The composition according to claim 4, wherein said composition contains the at least one strain of bacteria in an amount comprised from 0.1 to 65% by weight, relative to the total weight of the composition.

14. The method of claim 1, wherein the anti-inflammatory cytokine is IL-4, the first proinflammatory cytokine is IL-12p70, the second proinflammatory cytokine is IFN gamma, and the third proinflammatory cytokine is IL-17.

15. The method of claim 3, wherein the one or more strains are selected from the group consisting of Bifidobacterium longum DLBL 07 DSM 25669, Bifidobacterium longum DLBL 08 DSM 25670, Bifidobacterium longum DLBL 09 DSM 25671, Bifidobacterium longum DLBL 10 DSM 25672, and Bifidobacterium longum DLBL 11 DSM 25673.

16. The method of claim 7, wherein the uremic toxins are indole or cresol.

17. The composition of claim 9, wherein the Lactobacillus buchneri Lb 26 (DSM 16341) and/or Bifidobacterium lactis Bb1 (DSM 17850) are in a tyndallized form.

18. The composition of claim 4, wherein the composition has a bacterial load from 1×10⁸ to 1×10¹⁰ UFC/g.

19. The composition of claim 4, wherein the composition contains the at least one strain of bacteria in an amount from 0.5 to 15% by weight relative to the total weight of the composition.

20. The composition of claim 4, wherein the composition contains the at least one strain of bacteria in an amount from 1 to 10% by weight relative to the total weight of the composition.
